(19) United States
(12) Patent Application Publication
(10) Pub. No.: US 2010/0087650 A1
(43) Pub. Date: Apr. 8, 2010

(54) (1R,1'R)-ATRACURIUM SALTS SEPARATION PROCESS

(75) Inventors: Elena Ostrovsky, Rishon Le-Zion (IL); Oded Arad, Rehovot (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CHEMAGIS LTD., Bnei Brak (IL)

(21) Appl. No.: 12/529,771

(22) PCT Filed: Mar. 5, 2008
(86) PCT No.: PCT/IL2008/000291

§ 371 Date: Dec. 16, 2009

Related U.S. Application Data
(60) Provisional application No. 60/908,088, filed on Mar. 26, 2007.

(51) Int. Cl. C07D 401/12

(52) U.S. Cl. 546/140

(57) ABSTRACT

Provided is a method for separating cisatracurium from a mixture of atracurium isomers, which method includes eluting from a Reverse Phase (RP) stationary phase with a mobile phase in which the isomers are stable. The method of the present invention can be conveniently and inexpensively scaled up.

### TECHNICAL FIELD

The present invention relates to chromatography and more particularly to an improved method of separating the (1R,1′R)-atracurium salts isomers by means of high pressure liquid chromatography (HPLC).

### BACKGROUND OF THE INVENTION

Neuromuscular blocking agents (e.g., atracurium besylate, pancuronium bromide, rocuronium bromide, vecuronium bromide) are known to have muscle paralyzing activity that is similar to the alkaloid curare or d-tubocurarine. Neuromuscular blocking agents interrupt transmission of nerve impulses at the skeletal neuromuscular junction and are typically divided into two types: competitive, stabilizing blockers (non-depolarizing neuromuscular agents) and noncompetitive, depolarizing agents (depolarizing neuromuscular agents). Both types prevent acetylcholine from triggering the muscle contraction and are typically used as anesthesia adjuvants in the operating theatre for aiding intubation i.e. relaxation of vocal cords, trachea, jaw muscles etc and also for surgery i.e. providing generalized muscle relaxation, as relaxants during electroshock, in convulsive states, etc. Typically, therapy is performed by i.v. administration of a suitable dosage form.

Atracurium besylate [2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate] was first approved for human medical use in 1982. The isomer 1R-cis-1′R-cis-2,2′2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate is named cisatracurium besylate and it is 3-4 times more potent than atracurium besylate itself. The product was launched by GSK and Abbott Laboratories in 1996 under the trade name NIMBEX®. NIMBEX® is a sterile, non-pyrogenic aqueous solution that is adjusted to pH 3.25 to 3.65 with benzenesulfonic acid. The drug is provided in 2.5 ml, 5 ml and 10 ml ampules having a strength of 2 mg/ml cisatracurium besylate. In addition, a 30 ml vial containing 5 mg/ml cisatracurium besylate is also available.

Cisatracurium besylate has the structural formula below.

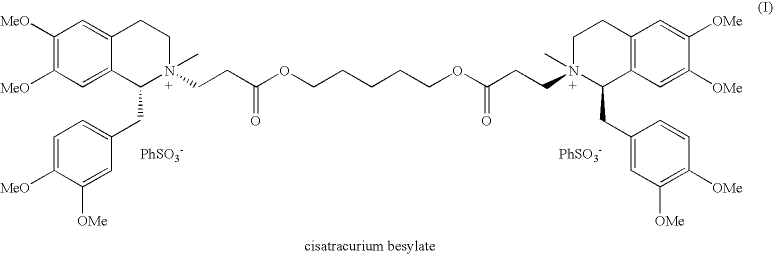

Because the atracurium compound has 4 chiral centers, there are theoretically 16 possible isomers. Due to symmetry of the molecule, the number of isomers is reduced to 10 (optical and geometrical isomers). This is discussed in detail by J. B. Stenlake et al. in “Biodegradable neuromuscular blocking agents,” Eur. J. Med. Chem.-Chem. Ther., 19, No. 5, pages 441-450 (1984).

The preparation of cisatracurium besylate is described in U.S. Pat. Nos. 5,453,510, and 5,556,987 and is depicted in Scheme 1 below. The process involves reacting 1,5-pentanediol with 3-bromopropionic acid in toluene with a trace of p-toluenesulfonic acid to afford 1,5-pentamethylene diacrylate. (±)-terahydropapaverine hydrochloride is resolved with N-acetyl-L-leucine to obtain (R)-terahydropapaverine N-acetyl-L-leucinate, which is converted to the free base and reacted with the 1,5-pentamethylene diacrylate in hot glacial acetic acid, purified by column chromatography, and treated with oxalic acid to afford (1R,1′R)-2,2′-(3,11-dioxo-4,10-dioxatridecylene)-bis-(1,2,3,4-tetrahydro-6,7-dimethoxy-2-methyl-1-veratryliso-quinolinium dioxalate (II), which is treated with methyl benzenesulfonate to obtain the (1R,1′R)-atracurium besylate isomer mixture, i.e., (1R-cis-1′R-cis), (1R-cis-1′R-trans) and (1R-trans-1′R-trans) isomers in a ratio of 58:34:6 respectively. The mixture is separated by means of liquid chromatography, using either irregular or spherical silica column in a mixture of dichloromethane and a strong acid, e.g., methanesulfonic acid, or in a mixture of dichloromethane, methanol and a strong acid, e.g., benzenesulfonic acid.

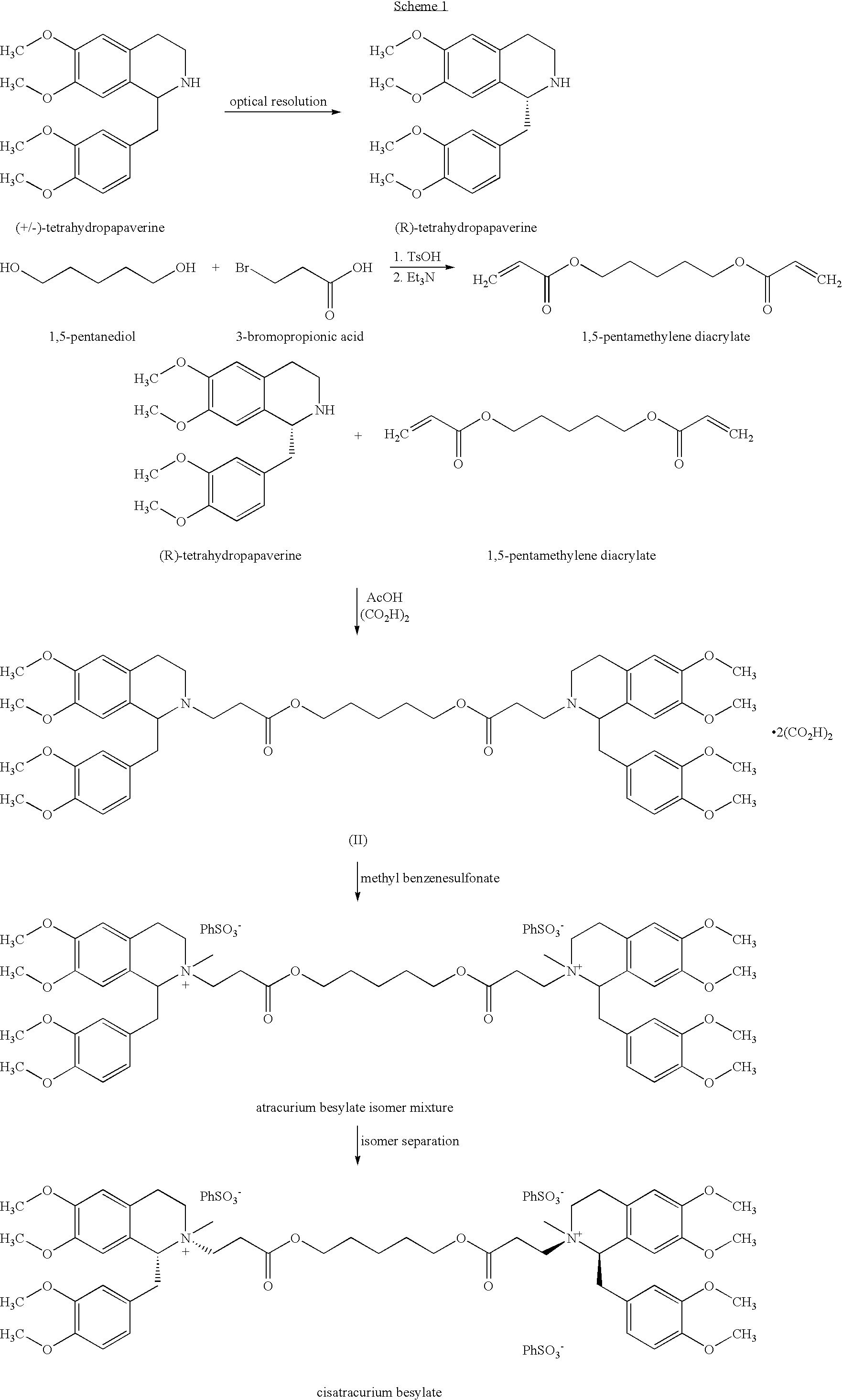

U.S. Pat. No. 5,453,510 (column 1, lines 31-39) teaches that aqueous mobile phases do not allow the recovery of the isomers of (1R,1′R)-atracurium without substantial degradation of the product. The instability of (1R,1′R)-atracurium isomers in aqueous mobile phases is problematic and precludes the use of methods that might otherwise be desirable for purifying cisatracurium besylate, particularly on a commercial scale. Furthermore, HPLC methods for separating the isomers of (1R,1′R)-atracurium besylate using strong acids may be unsatisfactory for large scale production because stainless steel (commonly used in HPLC instruments) is not compatible with strong acids (such as benzenesulfonic acid) due to an excessive corrosion of stainless steel components resulting in the possible contamination of the product, which is also undesirable, especially on large scale. Accordingly, there is a need for an improved method for separating the isomers of (1R,1′R)-atracurium besylate, particularly a commercially viable method, which employs an aqueous mobile phase and yet avoids problems associated with aqueous mobile phase instability. The present invention provides such methods.

### BRIEF SUMMARY OF THE INVENTION

In one embodiment, the present invention provides a chromatographic method for separating the (1R,1′R)-atracurium salt (e.g., the besylate salt) isomer mixture, which includes, e.g., Reverse Phase, High Performance Liquid Chromatography (HPLC), to produce highly pure 1R-cis,1′R-cis isomer (cisatracurium besylate). In accordance with the present invention, the desired 1R-cis,1′R-cis isomer can be separated from the (1R,1′R)-atracurium salt (e.g., the besylate salt) isomer mixture by:

loading a solution of (1R,1′R)-atracurium salt (e.g., the besylate salt) into an HPLC system equipped with a separating column comprising a suitable Reverse Phase (RP) stationary phase;

eluting the column with an eluent, which includes an aqueous phase, an organic solvent or a mixture thereof;

collecting at least one fraction comprising the desired product; and

isolating the product.

Suitable RP stationary phases can include, for example, C1 stationary phase, C3 stationary phase, C4 stationary phase, C8 stationary phase, C14 stationary phase, C18 stationary phase, other polymeric packing, e.g., polyamide, polymethacrylate, polystyrene, and the like.

Thus, a preferred method of the invention includes performing HPLC separation using a C18 RP stationary phase, which is eluted with a mobile phase comprising a buffer, that is, a mixture of a weak acid and its conjugate salt (e.g., acetic acid and sodium acetate, citric acid and sodium citrate, or ammonium formate and formic acid) and a solvent such as methanol.

### BRIEF DESCRIPTION OF THE DRAWINGS

FIG. 1 depicts the effect of buffer concentration on the retention of the 1R-cis,1′R-cis isomer

FIG. 2 depicts the effect of buffer concentration on the resolution of the 1R-cis,1′R-trans and 1R-cis,1′R-cis isomers

FIG. 3A depicts the chromatogram of an atracurium besylate reference sample.

FIG. 3B lists peak data associated with the chromatogram depicted in FIG. 3A.

FIG. 4A depicts the chromatogram of atracurium besylate, sample 1.

FIG. 4B lists peak data associated with the chromatogram depicted in FIG. 4A.

FIG. 5A depicts the chromatogram of cisatracurium besylate, sample 2.

FIG. 5B lists peak data associated with the chromatogram depicted in FIG. 5A.

FIG. 6A depicts the chromatogram of (1R,1′R)-atracurium besylate obtained according to the gradient detailed in Table 3.

FIG. 6B lists peak data associated with the chromatogram depicted in FIG. 6A.

FIG. 7 depicts the stability of (1R,1′R)-atracurium besylate at different pH values.

### DETAILED DESCRIPTION OF THE INVENTION

Contrary to the teaching of U.S. Pat. No. 5,453,510, it has been surprisingly found by the inventors of the present invention that Reverse Phase (RP) High Performance Liquid Chromatography (HPLC) column chromatography methods can be applied for separating the isomers of (1R,1′R)-atracurium salt (e.g., the besylate salt) and for obtaining the 1R-cis,1′R-cis isomer in highly pure form, using a mildly acidic or buffered mobile phase, e.g., mobile phases containing a solvent and an aqueous phase, which can include a weak acid or a buffer, e.g., a mixture of a weak acid such as acetic acid and its conjugate salt such as sodium acetate. Thus, using a mobile phase containing a solvent and an mildly acidic or buffered aqueous phase enables separating the isomers at conditions in which the 1R-cis,1′R-cis isomer is stable.

The term “mildly acidic” mobile phase, as described herein, refers to mobile phase containing a solvent and an aqueous phase, which includes a weak organic acid, having pKa value of 2.5 and higher, such as acetic acid. The “mildly acidic” mobile phase is distinguishable from the methods described above, using strong acids (having pKa value of 0 and lower) such as benzenesulfonic acid.

The term “buffered” mobile phase, as described herein, refers to a mobile phases containing an aqueous phase, which includes a weak organic acid and its conjugate salt, such as formic acid and ammonium formate

As used herein the term “substantially free of other geometrical and optical isomers” means that no other geometrical and optical can be detected within the limits of the HPLC method.

The term “isomeric purity” as defined herein, refers to the area percent of the peak corresponding to the 1R-cis,1′R-cis isomer relative to the area percent of the (1R-cis,1′R-cis isomer), (1R-cis,1′R-trans) and (1R-trans,1′R-trans) isomers. Isomeric purity may be expressed in terms of the following equation:

$$Isomeric purity = \frac{A}{A + B + C} \times 100$$

wherein, A=% area of the 1R-cis,1′R-cis isomer, B=% area of the 1R-cis,1′R-trans isomer and C=% area of the 1R-trans,1′R-trans isomer.

Thus, the present invention provides a chromatographic method for separating the isomers of (1R,1′R)-atracurium salt (e.g., the besylate salt), which uses a Reverse Phase HPLC (RP HPLC) method for obtaining a highly pure product, that is 1R-cis,1′R-cis isomer salt (e.g., the besylate salt), having isomeric purity higher than 99.5%.

According to one embodiment of the present invention, separating the desired 1R-cis,1R′-cis isomer from a mixture of isomers by a method, which includes:

loading a solution of (1R,1′R)-atracurium salt (e.g., the besylate salt) into a Reverse Phase HPLC system, equipped with a suitable separating column comprising a stationary phase;

eluting the column with an eluent mobile phase that includes an aqueous phase, an organic solvent or a mixture thereof;

collecting at least one fraction comprising the desired product; and

isolating the product.

The 1R-cis,1′R-cis isomer obtained in accordance with the present invention preferably is substantially free of other geometrical and optical isomers. As is evident from the experimental section of the present application, the obtained 1R-cis,1′R-cis isomer salt (e.g., the besylate salt) contains less than about 1% of the 1R-trans,1′R trans and/or 1R-cis,1′R-trans isomer, preferably less about than 0.1% of the 1R-trans,1′R-trans and/or 1R-cis,1′R-trans isomer. Thus, the highly pure 1R-cis,1′R-cis isomer is obtained having isomer purity of at least about 98.5%, and preferably having an isomer purity higher than 99.5%.

In accordance with the present invention, a suitable RP stationary phase column can include C1 stationary phase, C3 stationary phase, C4 stationary phase, C8 stationary phase, C14 stationary phase, C18 stationary phase, other polymeric packing, e.g., polyamide, polymethacrylate, polystyrene, and the like.

The RP HPLC separation method of the present invention can be utilized on analytical, semi-preparative and preparative scales. Preferred organic bonded reverse phases for obtaining the 1R-cis-1′R-cis isomer include C1, C4 and C18 phases. The results of exemplary RP methods for isolating the 1R-cis-1′R-cis isomer from the (1R,1′R)-atracurium besylate isomer mixture on different stationary phases are provided in Table 1 below.

TABLE 1

| Stationary phase | Result |
|---|---|
| C18 | Complete separation of all the isomers |
| C4 | Complete separation of all the isomers |
| C1 | Complete separation of all the isomers |
| CN | Poor separation of the 1R-cis-1′R-cis isomer |
| | from the 1R-cis,1′R-trans isomer, good |
| | separation of the 1R-trans,1′R-trans isomer |
| Phenyl | No separation of the 1R-cis,1′R-cis isomer |
| | from the 1R-cis,1′R-trans isomer, good |
| | separation of the 1R-trans,1′R-trans isomer |

According to a preferred embodiment of the present invention, the eluent includes an aqueous phase that contains at least one organic solvent. The aqueous phase preferably comprises an aqueous mixture of an acid and optionally also an inorganic salt (e.g. NaCl) or an amine (e.g., triethylamine).

In a preferred embodiment, the aqueous phase further includes a buffer, e.g., a mixture of a weak acid and its conjugate salt (e.g., acetic acid and sodium acetate or citric acid and sodium citrate). Suitable buffers include, for example, mixtures of acetic acid and sodium acetate, citric acid and sodium citrate, formic acid and ammonium formate, and the like. The acid can be either an organic or inorganic acid. Preferred organic acids include, for example, acetic acid, citric acid, formic acid, camphoric acid, adamantaneacetic acid and the like, and combinations thereof. Preferred inorganic acids include, for example, hydrochloric acid, hydrobromic acid, phosphoric acid, boric acid, nitric acid, and the like, and combinations thereof.

Suitable organic solvents, which may be included in the aqueous mobile phase in accordance with the present invention, include, for example, acetonitrile, methanol, ethanol, isopropyl alcohol, tetrahydrofuran (THF), and the like, and mixtures thereof.

In one embodiment, the (1R,1′R)-atracurium besylate isomers are separated by eluting with an aqueous phase that contains a salt of an acid, or an acid and a conjugate salt of an acid (e.g., nitric acid and sodium nitrate), at a pH of from about 1.0 to about 5.5, and more preferably at a pH of from about 3.0 to about 3.5. Suitable acid salts can include, for example, ammonium formate, sodium formate, ammonium acetate, sodium acetate, sodium nitrate, sodium chloride, potassium chloride, barium chloride, sodium bromide, calcium bromide, monopotassium dihydrogenphosphate, monosodium dihydrogenphosphate, and the like, and combinations thereof.

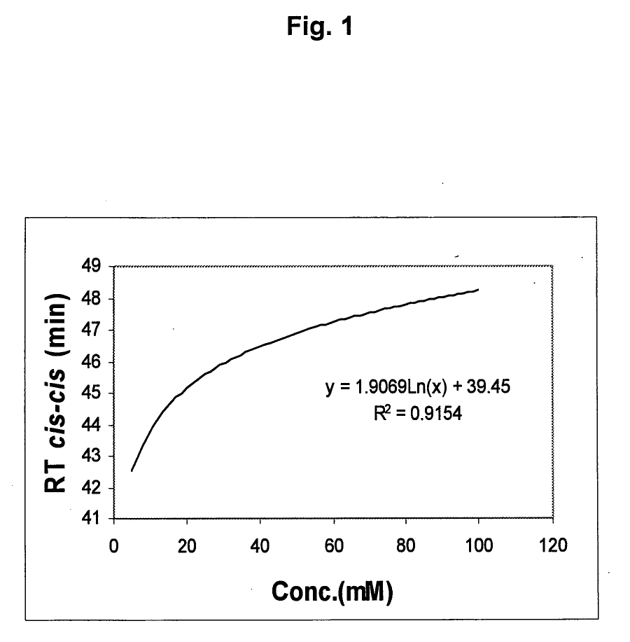

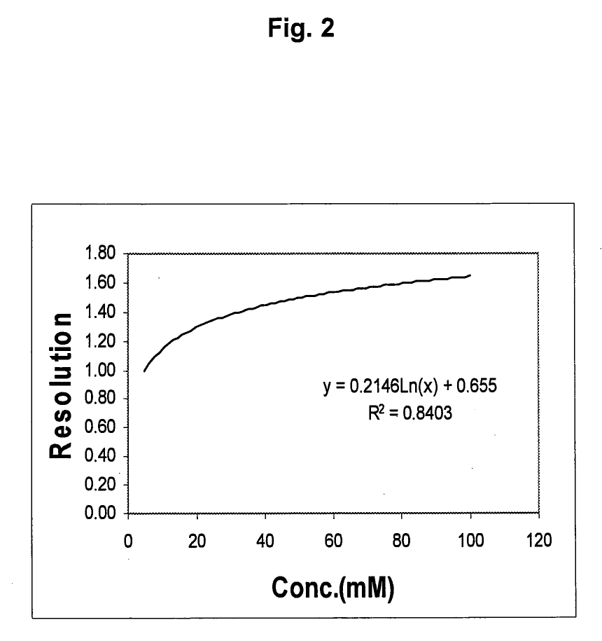

Preferred buffer concentrations, for isolating the 1R-cis,1′R-cis isomer, range from about 20 mM to about 40 mM. In some instances, a buffer concentration in the higher range increases the retention time of the 1R-cis,1′R-cis isomer, as depicted in FIG. 1, and also improves the isomer's separation, as depicted in FIG. 2.

The cisatracurium salt is substantially stable under the separation conditions of the present invention. For instance, the extent of cisatracurium degradation is only slightly (less than 2%) after 26 hours at room temperature in a solvent mixture, e.g., at pH 2, using nitrate buffer, and almost no degradation (0.2%) was observed after 26 hours in a solvent mixture at 4° C. using a nitrate buffer (see Example 6, Table 12).

A desired counter anion may be introduced, e.g., by an ion exchange process, which can include, e.g., adding desired counter anions to the system to exchange with other anions utilized in the separation process, which may associate with one or more of the (1R,1′R)-atracurium isomers. Any suitable ion exchange methods may be utilized as well, e.g., a suitable ion exchange resin.

The isolation of the cisatracurium from the elution liquid mixture can be carried out by any suitable method such as Solid Phase Extraction (SPE). A non-limiting example of a method for isolating the cisatracurium solution from the elution liquid is by SPE. For example, the isolation can be effected by successively transferring a cisatracurium besylate sample solution (containing the aqueous phase and the organic solvent) and water through a C18 sorbent, which causes the separation of the cisatracurium besylate from the buffer species. The cisatracurium besylate retained by the sorbent can be then removed, e.g., by eluting from the sorbent with methanol. The evaluation of the anions is preferably carried out by HPLC, and the cations are preferably evaluated indirectly (since the cations and the anions are removed at about the same rate). Recovery of the isolated material and the anions can be checked after performing each step of the SPE method.

Thus, the present invention provides a SPE method for isolating a product (e.g., an isomer of (1R,1′R)-actracurium besylate) from an HPLC elution liquid mixture, which method preferably includes:

successively transferring the sample solution and water through a sorbent;

eluting the product from the sorbent with an organic solvent, e.g., methanol; and

washing the sorbent with an organic solvent, e.g., methanol.

The SPE method of the present invention can be utilized for isolating the 1R-cis,1′R-cis isomer from a HPLC elution liquid mixture thereof. In one embodiment, the method includes:

optionally evaporating at least a portion of the organic solvent from an aqueous HPLC elution liquid containing an organic solvent;

adding an organic solvent and separating the phases and optionally washing the organic layer;

optionally changing the anion using a suitable ion exchange method; and

isolating the product from the aqueous phase by spray-drying or freeze-drying.

The organic solvent added in the SPE method of the present invention can include, for example, ethyl acetate, n-propyl acetate, isopropyl acetate, n-butyl acetate, isobutyl acetate, toluene, chloroform, dichloromethane, and the like, and mixtures thereof. A preferred solvent for the SPE method of the present invention is dichloromethane.

The present invention further provides a pharmaceutical composition comprising substantially pure cisatracurium besylate, which can be obtained in accordance with the present invention, and can be employed, e.g., in surgery as a neuromuscular blocking agent as a formulation for administration, e.g., by injection or infusion.

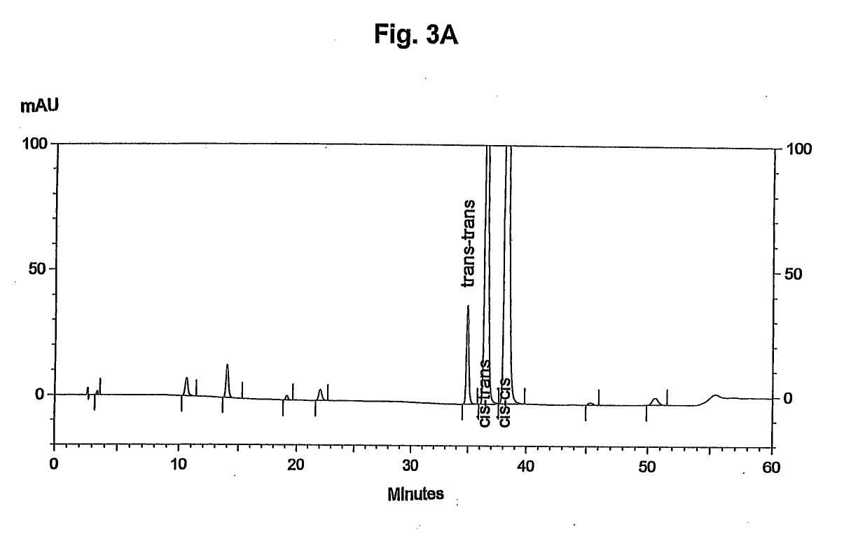

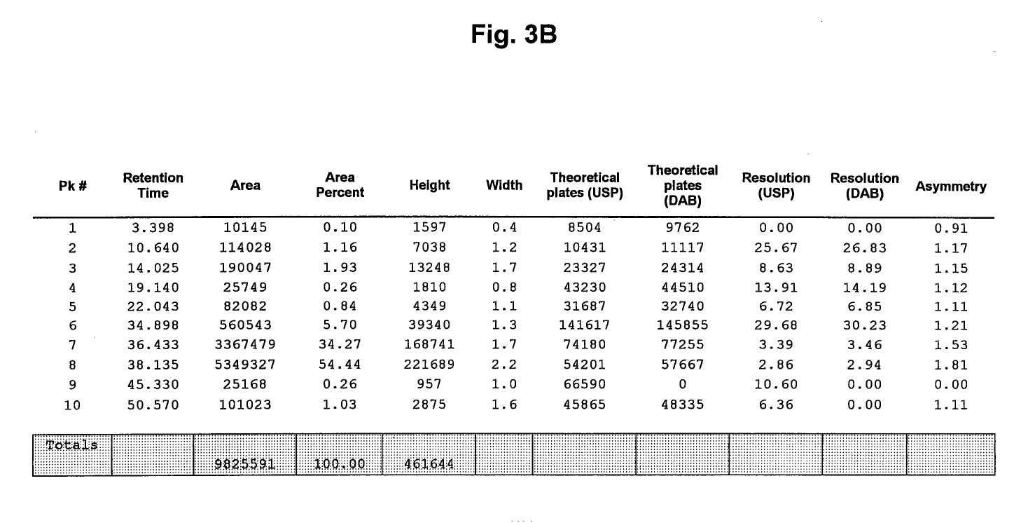

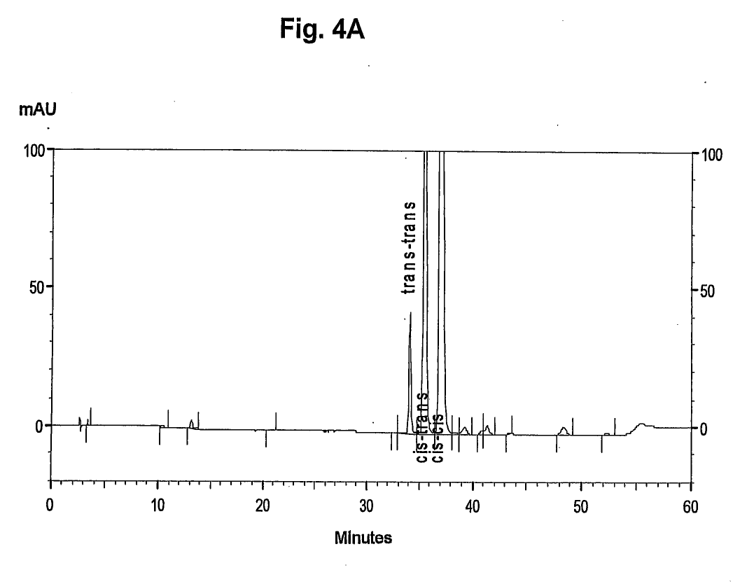

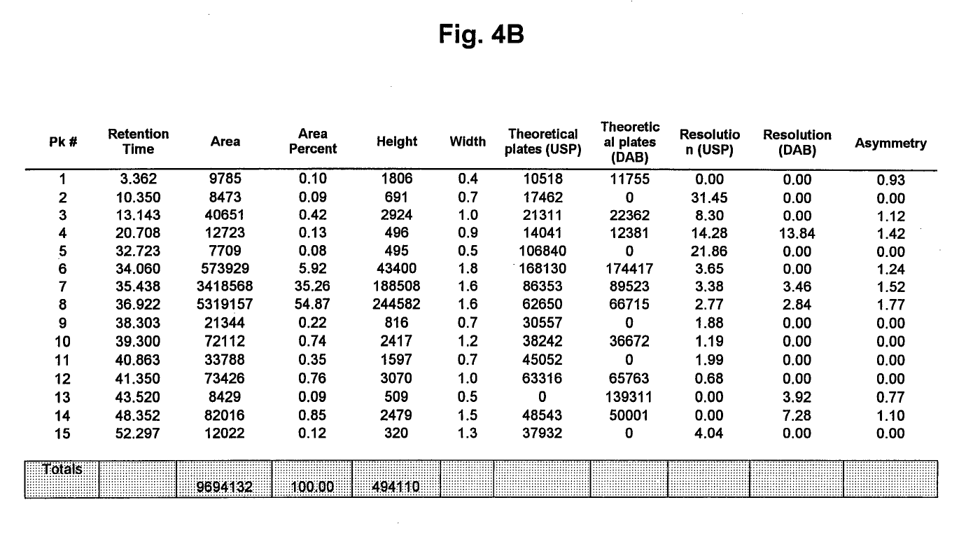

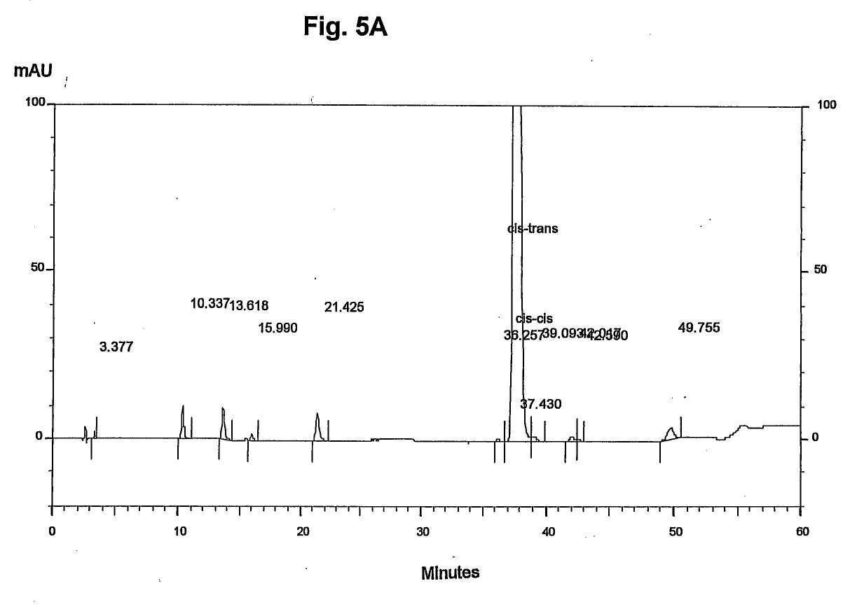

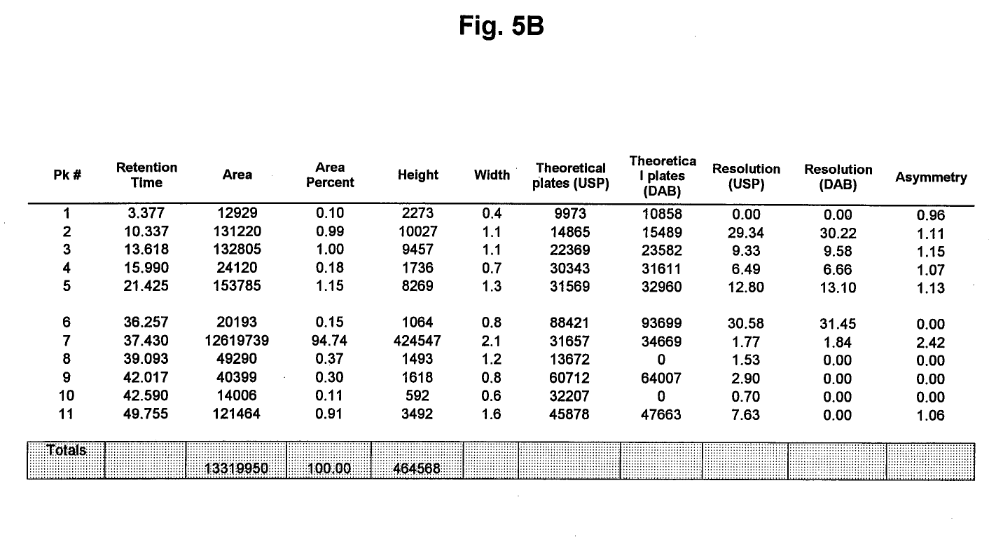

FIG. 3 depicts the chromatogram of a reference sample obtained by diluting a lyophilized atracurium besylate sample, containing, inter alia, approximately 54% 1R-cis,1′R-cis, 34% 1R-cis,1′R-trans and 6% 1R-trans,1′R-trans isomers (hereinafter the “reference sample”), and a small amount of benzenesulfonic acid in the corresponding aqueous phase, and injecting the mixture into the HPLC system, equipped with a C18 stationary phase according to a gradient method in which the eluent contains a mixture of an aqueous phase and at least one solvent. FIG. 4 depicts the chromatogram of an exemplary starting sample of (1R,1′R)-atracurium besylate isomer mixture (hereinafter “sample 1”), and FIG. 5 depicts the chromatogram of a cisatracurium besylate (NIMBEX®) buffer solution (hereinafter “sample 2”).

The following examples further illustrate the invention but, of course, should not be construed as in any way limiting its scope.

### EXAMPLES

Cisatracurium besylate was analyzed by High Performance Liquid Chromatography (HPLC). Exemplary HPLC separations were performed, e.g., using the following conditions: Column and packing—Hypersil Hyperprep HS C18, 250×21.2 mm, 15μ, P.N. 37115-125; UV detection—UV operated at 280 nm; flow rate: 13 ml/min; Mobile phase: Eluent A: 20 mM NaNO₃, pH adjusted to 2.0 with HNO₃. Eluent B: methanol. The gradient elution is as detailed in Table 2:

TABLE 2

| Time, minutes | % eluent A | % eluent B |
|---|---|---|
| 0 | 60 | 40 |
| 65 | 60 | 40 |
| 67 | 55 | 45 |
| 90 | 55 | 45 |

Another example of HPLC separation using the same column as in the previous example is: UV detection at 280 nm; flow rate: 13 ml/min; Mobile phase: Eluent A: 40 mM buffer solution containing diethylamine (DEA), pH adjusted to 3.5 with formic acid Eluent B: methanol. The gradient elution is as detailed in Table 3:

TABLE 3

| Time, minutes | % eluent A | % eluent B |
|---|---|---|
| 0 | 65 | 35 |
| 70 | 65 | 35 |
| 72 | 55 | 45 |
| 90 | 55 | 45 |

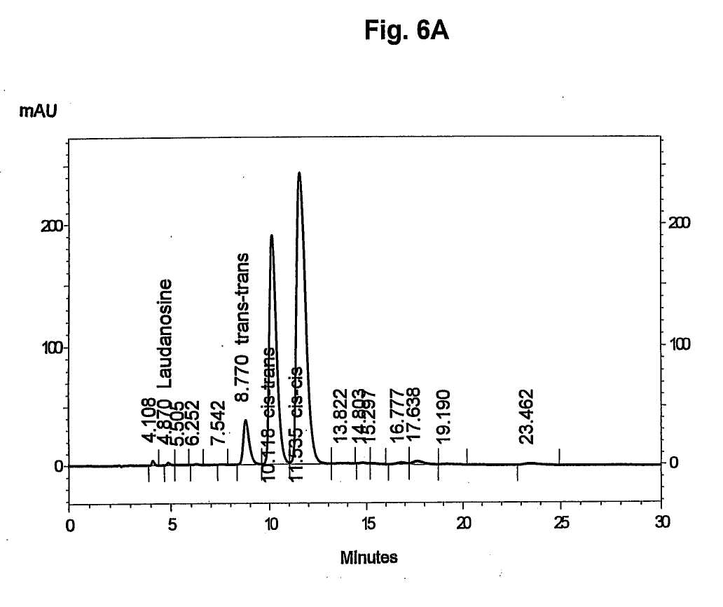

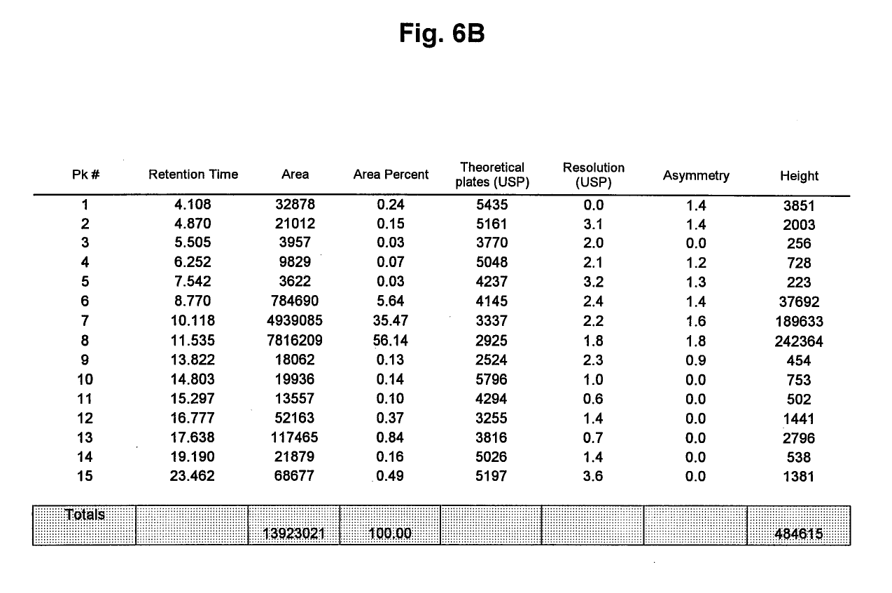

The chromatogram corresponding to the separation of (1R,1′R)-atracurium besylate, according to the method using the gradient detailed in Table 3, is depicted in FIG. 6.

### Example 1

This example demonstrates a Reverse Phase procedure for isolating cisatracurium besylate on C18 stationary phase using different aqueous phases.

Atracurium besylate reference sample (10 mg/ml) was analyzed on a C18 stationary phase by gradient elution, using different aqueous phases in the solvent mixtures with methanol. Column and packing: Alltech, Altima C18, 250×4.6×5μ, Cat. No. 88056; UV detection: 280 nm; flow rate: 1 ml/min; The results, including the gradient elutions, are detailed in Table 4:

TABLE 4

| | | Gradient | | | Result* | | |
|---|---|---|---|---|---|---|---|
| No | Aqueous phase | Time | % buffer | % MeOH | Isomer | RT | Resolution |
| 1 | Aqueous solution at | 0 | 65 | 35 | trans-trans | 26.4 | |
| | pH = 3.0 with | 20 | 65 | 35 | cis-trans | 29.6 | 1.8 |
| | trifluoroacetic acid | 25 | 60 | 40 | cis-cis | 33.0 | 1.5 |
| | (TFA) | 60 | 60 | 40 | | | |
| | | 61 | 65 | 35 | | | |
| | | 70 | 65 | 35 | | | |
| 2 | 10 ml of | 0 | 65 | 35 | trans-trans | 45.5 | |
| | triethylamine (TEA) | 20 | 65 | 35 | cis-trans | 51.8 | 3.7 |
| | in 1 L of water, | 25 | 55 | 45 | cis-cis | 59.5 | >4.5 |
| | pH = 3.0, with TFA | 60 | 55 | 45 | | | |
| | | 61 | 65 | 35 | | | |
| | | 70 | 65 | 35 | | | |
| 3 | Aqueous solution at | 0 | 80 | 20 | No separation between the | | |
| | pH = 3.0 | 30 | 60 | 40 | isomers (RT's = 27-30 min) | | |
| | (with acetic acid) | 65 | 60 | 40 | | | |
| | | 61 | 80 | 20 | | | |
| | | 70 | 80 | 20 | | | |
| 4 | 2 ml TEA in 1 L of | 0 | 70 | 30 | trans-trans | 35.5 | |
| | water at pH = 3.5 | 30 | 60 | 40 | cis-trans | 37.8 | >2.0 |
| | (with acetic acid) | 60 | 60 | 40 | cis-cis | 41.0 | >1.5 |
| | | 61 | 70 | 30 | | | |
| | | 70 | 70 | 30 | | | |
| 5 | 6.45 g sodium citrate + | Same gradient as in | | | trans-trans | 31.2 | |
| | 5 g citric acid in 1 L | experiment No. 4 | | | cis-trans | 34.5 | 2.5 |
| | of water at pH = 3.5 | | | | cis-cis | 38.4 | 1.9 |
| 6 | Aqueous solution at | 0 | 75 | 25 | No separation between the | | |
| | pH = 3.0 | 30 | 65 | 35 | isomers (RT's = 20-26 min) | | |
| | (with formic acid) | 60 | 65 | 35 | | | |
| | | 61 | 75 | 25 | | | |
| | | 70 | 75 | 25 | | | |
| 7 | 1.26 g ammonium | 0 | 70 | 30 | trans-trans | 29.0 | |
| | formate (0.02M) in | 30 | 60 | 40 | cis-trans | 32.1 | >2.5 |
| | 1 L of water at | 60 | 60 | 40 | cis-cis | 35.5 | ~2.0 |
| | pH = 3.0 | 61 | 70 | 30 | | | |
| | (with formic acid) | 70 | 70 | 30 | | | |

| 8 | Aqueous solution of | Same gradient as in | No separation between the | | |
|---|---|---|---|---|---|
| | pH = 3.0 (with HCl) | experiment No. 7 | isomers (RT's = 10-20 min) | | |
| 9 | 1.25 g NaCl in 1 L of | Same gradient as in | trans-trans | 32.1 | |
| | water (0.02M), | experiment No. 7 | cis-trans | 35.5 | 2.0 |
| | pH = 3.0 (with HCl) | | cis-cis | 40.0 | 1.6 |
| 10 | 4.16 g BaCl₂ (0.02M | Same gradient as in | trans-trans | 33.3 | |
| | Ba⁺²) in 1 L of water | experiment No. 7 | cis-trans | 36.9 | >2.0 |
| | at pH = 3.0 (with HCl) | | cis-cis | 41.6 | >2.0 |

*The elution order of the atracurium isomers on the C18 phase is the following: trans-trans, cis-trans, cis-cis, RT = Retention Time (minutes)
Resolution = 2(t₂ − t₁)/w₁ + w₂), wherein t₁, t₂ are retention times (RT) of the eluted peaks and w₁, w₂ are the corresponding widths at the bases of the peaks obtained by extrapolating the relatively straight sides of the peaks to the baseline.

### Example 2

This example demonstrates the comparison of acetate and formate buffers for RP chromatographic separations of (1R,1′R)-atracurium besylate isomers.

(1R,1′R)-atracurium besylate finished dosage form (the reference sample), having concentration of 10 mg/ml, was analyzed on a C18 stationary phase by gradient elution, using a mixture of methanol and a buffer (pH 3.5). Two buffers were compared: acetate buffer and formate buffer (both of them are prepared using the corresponding ammonia salt). The results are presented in Table 5.

TABLE 5

| Acetate buffer | | | | Formate buffer | | | |
|---|---|---|---|---|---|---|---|
| Isomer | RT, min | Resolution | % Area | Isomer | RT | Resolution | % Area |
| trans-trans | 40.5 | | 5.8 | trans-trans | 20.1 | | 5.8 |
| cis-trans | 46.1 | 1.5 | 34.9 | cis-trans | 29.2 | 1.5 | 34.5 |
| cis-cis | 54.3 | 1.2 | 55.1 | cis-cis | 32.6 | 1.2 | 54.5 |

min = minutes

### Example 3

This example demonstrates the effect of pH on RP chromatographic separations of (1R,1′R)-atracurium besylate isomers.

(1R,1′R)-atracurium besylate finished dosage form (the reference sample), having concentration of 10 mg/ml, was analyzed on a C18 stationary phase by gradient elution using a mixture of methanol and a buffer. The pH of the ammonium acetate buffer (20 mM) was varied from 3.0 to 5.5. The results are depicted in Table 6.

TABLE 6

| | RT trans-trans | RT cis-trans | RT of cis-cis | Resolution of the cis- |
|---|---|---|---|---|
| pH | isomer, (min) | isomer, (min) | isomer, (min) | trans and cis-cis isomers |
| 3.0 | 19.1 | 21.4 | 24.3 | 1.0 |
| 3.5 | 24.5 | 27.2 | 30.5 | 1.1 |
| 4.0 | 26.5 | 29.4 | 32.8 | 1.1 |
| 4.5 | 27.1 | 30.0 | 33.4 | 1.1 |
| 5.5 | 30.2 | 32.9 | 35.9 | 1.2 |

min = minutes

### Example 4

This example demonstrates the effect of the buffer salt concentration on RP chromatographic separation of (1R,1′R)-atracurium besylate isomers.

(1R,1′R)-Atracurium besylate finished dosage form (the reference sample), having concentration of 10 mg/ml, was analyzed on a C18 stationary phase by gradient elution of methanol buffer. The concentration of ammonium acetate buffer (pH=3.5) was varied from 5 mM to 100 mM. The results are depicted in Table 7.

TABLE 7

| Concentration of | RT of trans- | RT of cis- | RT of cis- | Resolution of the cis- |
|---|---|---|---|---|
| CH₃COO⁻ NH4⁺ (mM) | trans, (min) | trans, (min) | cis (min) | trans and cis-cis isomers |
| 5.0 | 36.9 | 39.3 | 41.8 | 0.9 |
| 10.0 | 39.7 | 41.8 | 44.1 | 1.2 |
| 20.0 | 41.6 | 44.0 | 46.1 | 1.4 |
| 100.0 | 43.1 | 45.6 | 47.7 | 1.6 |

min = minutes

### Example 5

This example demonstrates the separation of the (1R,1′R)-atracurium besylate reference sample.

The (1R,1′R)-atracurium besylate reference sample was separated by semi-preparative Reverse Phase HPLC method as follows: Hypersil Hyperprep HS C18, 250×21.2 mm, 15μ Column, conditions: 20 mM NaNO₃, pH adjusted to 2.0 with HNO₃. Eluent B: methanol. Flow rate: 13 ml/minute. The gradient elution is as detailed in Table 2.

The sample solutions for the preparative HPLC separation were prepared as follows: Solution 1, 827.3 mg of the (1R,1′R)-atracurium besylate reference sample was dissolved in 20 ml Eluent A (concentration: 33.1 mg/ml). Solution 2, 623.5 mg of the (1R,1′R)-atracurium besylate reference sample was dissolved in 20 ml Eluent A (concentration: 31.2 mg/ml). A (1R,1′R)-atracurium besylate reference sample, having concentration of 1.56 mg/ml was prepared and kept cold for use in the identification and quantization of the isomers.

The sample solutions for preparative separation were loaded into the Reverse Phase C18 column. The column was eluted with 20 mM NaNO₃ solution (pH adjusted to 2.0 with HNO₃) and methanol. Table 8 summarizes the results of 11 runs of analyses of the combined fractions.

TABLE 8

| | Concentration | Total loading of | Total loading of | Total loading |
|---|---|---|---|---|
| | of the isomeric | the isomeric | the cis-cis | of the cis-cis |
| Run | mixture, mg/ml | mixture, mg | isomer, mg | cation, mg |
| 1 | 33.1 | 49.6 | 27.8 | 20.7 |
| 2 | 33.1 | 49.6 | 27.8 | 20.7 |
| 3 | 33.1 | 49.6 | 27.8 | 20.7 |
| 4 | 33.1 | 49.6 | 27.8 | 20.7 |
| 5 | 33.1 | 49.6 | 27.8 | 20.7 |
| 6 | 33.1 | 49.6 | 27.8 | 20.7 |
| 7 | 33.1 | 49.6 | 27.8 | 20.7 |
| 8 | 31.2 | 46.8 | 26.2 | 19.5 |
| 9 | 31.2 | 46.8 | 26.2 | 19.5 |
| 10 | 31.2 | 46.8 | 26.2 | 19.5 |
| 11 | 31.2 | 46.8 | 26.2 | 19.5 |
| Total | | 534.4 | 299.4 | 222.9 |

Fractions of the column eluate were collected and the fractions, containing the required 1R-cis,1′R-cis (cisatracurium) isomer, were combined and analyzed against the reference solution. Table 9 summarizes the results of analyses of the (1R,1′R)-atracurium besylate isomers. As indicated in Tables 9 and 10, the total loading of (1R,1′R)-atracurium besylate was 534.4 mg, while the total loading of cisatracurium besylate was 299.4 mg and the total loading of cisatracurium base was 222.9 mg (90% yield).

TABLE 9

| Fraction | Total | Total cisatracurium | % area of the | % area of the | amount of the |
|---|---|---|---|---|---|
| No. | volume, ml | base content, mg | cis-cis isomer | cis-trans isomer | cis-cis isomer, mg |
| 1 | 705 | 74.2 | 100.0 | 0.0 | 99.4 |
| 2 | 245 | 51.1 | 99.5 | 0.5 | 68.5 |
| 3 | 165 | 28.8 | 98.7 | 1.2 | 38.6 |
| 4 | 110 | 16.8 | 96.0 | 3.9 | 22.5 |
| 5 | 125 | 16.8 | 95.8 | 4.1 | 22.5 |
| 6 | 120 | 13.7 | 92.2 | 7.8 | 18.4 |
| 7 | 13 | 1.3 | 76.0 | 11.5 | 1.7 |
| Total | | 202.7 | | | 271.6 |

TABLE 10

| | | | Amount of the | Yield of the |
|---|---|---|---|---|
| | Isomeric | Total | cisatra-curium | cisatra-curium |
| No. | purity % | purity % | besylate, mg | besylate % |
| 1 | >99.5 | >99.5 | 167.8 | 56.0 |
| 2 | >98.5 | >98.5 | 38.6 | 12.9 |
| 3 | >95.5 | >95.5 | 45.0 | 15.0 |
| 4 | >92.0 | >92.0 | 18.4 | 6.1 |
| Total | | | 269.8 | 90.0 |

### Example 6

This example demonstrates the stability of (1R,1′R)-atracurium besylate in different buffers and at different temperatures.

The stability of the (1R,1′R)-atracurium besylate solution at room temperature for time periods of up to 24 hours was checked using different types of buffers (varying by the nature of the cation and the anion). The diluent was a mixture of 90% buffer and 10% methanol. The Cation concentration in each buffer was 20 mM. The HPLC conditions were according to the USP procedure. The results are depicted in Tables 11 and 12. The degradation (D), according to the data presented in Table 11, was calculated as follows:

$$D = \frac{{X}_{0} - {X}_{24}}{{X}_{0}} \times 100$$

wherein, X₀=% of cisatracurium at T₀, and X₂₄=% of cisatracurium at after 24 hours.

TABLE 11

| | | | % of cisatracurium | % of cisatracurium | % |
|---|---|---|---|---|---|
| No. | Buffer | pH | besylate at T = 0 | besylate after 24 hours | degradation |
| 1 | NH₄⁺CH₃COO⁻/CH₃COOH | 3.5 | 54.7 | 52.9 | 3.3 |
| 2 | NH₄⁺CH₃COO⁻/HCOOH | 3.5 | 54.6 | 54.3 | 0.5 |
| 3 | Na⁺CH₃COO⁻/CH₃COOH | 3.5 | 54.5 | 52.5 | 3.7 |
| 4 | Na⁺NO₃⁻/HNO₃ | 3.0 | 54.5 | 54.5 | 0 |
| 5 | K⁺H₂PO₄ | 3.0 | 54.7 | 53.7 | 1.8 |
| 6 | CaBr₂/HBr | 3.5 | 54.4 | 54.4 | 0 |
| 7 | diethylamine/CH₃COOH | 3.5 | 54.7 | 53.2 | 2.7 |
| 8 | triethylamine/CH₃COOH | 3.5 | 54.4 | 52.1 | 4.2 |

| | | |
|---|---|---|
| 9 | Na⁺ClO₄/HClO₄ | Atracurium besylate is precipitated in the |
| | | presence of a ClO₄ ion |

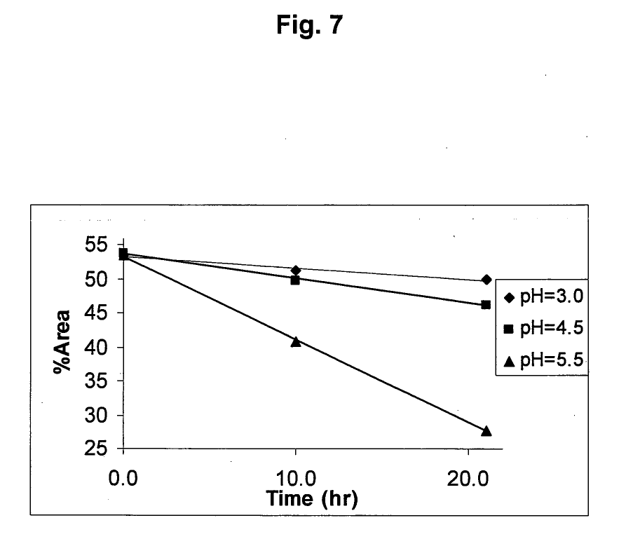

A graph depicting the stability of the 1R-cis, 1R′-trans isomer at different pH values is provided in FIG. 7, which demonstrates that at pH 3, after 20 hours the % area of the 1R-cis,1′R-cis isomer is only slightly reduced while at pH 5.5 the % area of the 1R-cis,1′R-cis isomer is significantly reduced.

The degradation (D) according to the data presented in Table 12 was calculated as follows:

$$D = \frac{{X}_{0} - {X}_{21}}{{X}_{0}} \times 100$$

wherein, X₀=% of cisatracurium at T₀, and X₂₁=% of cisatracurium at after 24 hours.

TABLE 12

| | pH | | | | | |
|---|---|---|---|---|---|---|
| Time, hours | 3.0 | 3.5 | 4.0 | 4.5 | 5.0 | 5.5 |
| 0.0 | 53.4 | 53.6 | 53.4 | 53.9 | 53.6 | 53.6 |
| 10.0 | 51.3 | 51.0 | 50.5 | 49.9 | 46.1 | 40.8 |
| 21.0 | 50.0 | 49.2 | 47.9 | 46.2 | 38.4 | 27.8 |
| % degradation* | 6.4 | 8.2 | 10.3 | 14.3 | 28.4 | 48.1 |

*The buffer used was the Na⁺CH₃COO⁻/CH₃COOH buffer at 3 different pH values, that is pH values of 3.0, 4.6 and 5.5. The values in the table are represented as % of cisatracurium besylate.

A sample solution of (1R,1′R)-atracurium besylate (10 mg/ml) was prepared using two buffer solutions at pH values of 1.0 and 2.0 and analyzed on the C18 stationary phase by gradient elution [20 mM KNO₃ buffer (at pH corresponding to sample preparation)—methanol]. The stability of the sample solution at the mentioned pH values was demonstrated at room temperature and at 4° C., as depicted in Table 13.

TABLE 13

| | % area of the cis-cis isomer | | | | | | | |
|---|---|---|---|---|---|---|---|---|
| Time | pH = 1 | | | | pH = 2 | | | |
| (hours) | RT | D, % | 4° C. | D, % | RT | D, % | 4° C. | D, % |
| 0 | 54.9 | | 54.9 | | 54.8 | | 54.8 | |
| 6 | 50.2 | 8.6 | 53.9 | 1.8 | 54.5 | 0.5 | 54.8 | 0 |
| 26 | 38.8 | 29.3 | 52.8 | 3.8 | 53.8 | 1.8 | 54.7 | 0.2 |

RT = room temperature, D = degradation

$$D = \frac{{X}_{0} - {X}_{6 / 26}}{{X}_{0}} \times 100$$

wherein, X₀=% of cisatracurium at T₀, and X_{6/26}=% of cisatracurium at after 6 or 26 hours.

### Example 7

This example demonstrates a method for purification of the cisatracurium solution from the buffer's mixture by Solid Phase Extraction (SPE).

A series of the sample solutions of (1R,1′R)-atracurium besylate isomer mixture (55% cis-cis; 35% cis-trans and 6% trans-trans isomer) was prepared in diluents containing different buffers (varying by the nature of the cation and the anion). The diluents consisted of a mixture of 90% buffer and 10% methanol. The sample solutions were purified using SPE C18 cartridge.

The evaluation of the buffer anions was carried out by HPLC. The cations were evaluated indirectly. The recovery of the isolate (1R cis,1′R-cis isomer) and anions was checked after each step of the SPE method, which comprises the steps of:

1) successive transferring of the sample solution and water through the sorbent;

2) elution of the sample with methanol; and

3) washing the sorbent with methanol.

The results of this study are summarized in the Table 14.

TABLE 14

| | | Anion removal (%) | | | Cis-cis isomer recovery (%) | | |
|---|---|---|---|---|---|---|---|
| No. | Buffer | Step 1 | Step 2 | Step 3 | Step 1 | Step 2 | Step 3 |
| 1 | 20 mM NH₄⁺CH₃COO⁻/ | 91.5 | 11.5 | 2.9 | 0.2 | 66.35 | 0.1 |
| | CH₃COOH, pH = 3.5 | | | | | | |
| 2 | 20 mM NH₄⁺COO⁻/ | 79.02 | 6.04 | 1.34 | 1.23 | 71.4 | 0.2 |
| | HCOOH, pH = 3.5 | | | | | | |
| 3 | 20 mM Na⁺CH₃COO⁻/ | 85.73 | 15.66 | ND | 10 | 90.8 | ND |
| | NaCOOH, pH = 3.5 | | | | | | |
| 4 | 20 mM Na⁺NO₃⁻/HNO₃, | NE | NE | NE | ND | 98.23 | 0.2 |
| | pH = 3.0 | | | | | | |
| 5 | 20 mM CaBr₂/HBr | 100.42 | 35 | ND | ND | 103.8 | 0.1 |
| | pH = 3.0 | | | | | | |

ND—Not detected, NE—Not evaluated

### Example 8

This example demonstrates a method of product isolation.

Fractions of column eluates containing the 1R-cis,1′R-cis isomer were collected manually via Hypersil Hyperprep HS C18 column, 250 mm*21.2 mm*15μ, P/N 37115-125, using the following eluents:

Eluent A: 20 mM NaNO₃ aqueous solution, pH adjusted to 2.0 with HNO₃
Eluent B: methanol.
The gradient was as described in Table 3, and the detection was at 280 nm. The flow rate was 14 ml/min and the cisatracurium besylate was isolated from the (1R,1′R)-atracurium besylate mixture and analyzed using an HPLC system. The Fractions were combined correspondingly to the 1R-cis,1′R-cis isomer content, as detailed in Table 15.

TABLE 15

| Conc. of | | | Loading weight of | |
|---|---|---|---|---|
| sample solution | Loading | Loading weight | the cis-cis isomer | % of cis- |
| (mg/ml) | volume (ml) | (mg/column) | (mg/column) | cis isomer |
| 30.7 | 1 | 30.7 | 17.2 | 99.1 |
| 30.7 | 1 | 30.7 | 17.2 | 99.6 |
| 30.7 | 2 | 61.4 | 34.4 | 99.6 |
| 30.7 | 2 | 61.4 | 34.4 | 99.5 |
| Total | | 184.2 | 103.2 | |

The fractions were combined (400 ml) and mixed with 200 ml of acidified brine (pH=2 with benzenesulfonic acid) and extracted with 150 ml dichloromethane (three consecutive extractions, 50 ml of dichloromethane each extraction). The organic phases were collected, dried with MgSO₄ and evaporated to dryness to afford residual semi-solid oil (91 mg), which was dissolved in 18 ml water and the pH was adjusted to ˜3 with benzenesulfonic acid. The aqueous solution was placed into the freeze dryer (in tree glass vials) for 40 hours. The aqueous solution was lyophilized to afford 72 mg of cisatracurium besylate in 60% yield, having purity of 96.3% (by HPLC).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for separating cisatracurium in high isomeric purity from (1R,1′R)-atracurium isomer mixture, which method comprises eluting the isomer mixture from a Reverse Phase (RP) stationary phase with a mildly acidic or buffered mobile phase, to separate the 1R-cis,1′R-cis isomer (cisatracurium) therefrom in high isomeric purity.

2. The method of claim 1, wherein the 1R-cis,1′R-cis isomer exhibits a degradation rate in the mobile phase of less than 2% after 26 hours at room temperature.

3. The method of claim 1, comprising:
loading a solution of a (1R,1′R)-atracurium salt isomer mixture into a reverse phase HPLC system equipped with a separating column and a reverse phase stationary phase;
eluting the column with an eluent, which includes an aqueous phase, an organic solvent or a mixture thereof, to separate a cisatracurium salt from the isomer mixture;
collecting at least one fraction comprising the cisatracurium salt; and
isolating the cisatracurium salt.

4. The method of claim 3, wherein the reverse phase stationary phase is a C1 stationary phase, a C3 stationary phase, a C4 stationary phase, a C8 stationary phase, a C14 stationary phase, a C18 stationary phase, or a polymeric packing.

5. The method of claim 4, wherein the reverse phase stationary phase is a C18 stationary phase.

6. The method of claim 3, wherein the mobile phase is an aqueous phase, which comprises an acid and, optionally, a salt or an amine.

7. The method of claim 6, wherein aqueous phase comprises a buffer, which a mixture of an acid and a conjugate salt thereof.

8. The method of claim 6, wherein the aqueous phase comprises a salt, which is ammonium formate, sodium formate, ammonium acetate, sodium acetate, sodium nitrate, sodium chloride, potassium chloride, barium chloride, sodium bromide, calcium bromide, monopotassium dihydrogenphosphate, monosodium dihydrogenphosphate, or a combination thereof.

9. The method of claim 6, wherein the acid is hydrochloric acid, hydrobromic acid, phosphoric acid, boric acid, nitric acid, or a combination thereof.

10. The method of claim 6, wherein the aqueous phase comprises nitric acid and sodium nitrate.

11. The method of claim 6, wherein the acid is acetic acid, citric acid, formic acid, camphoric acid, adamantaneacetic acid or a combination thereof.

12. The method of claim 7, wherein the buffer is a mixture of acetic acid and sodium acetate, a mixture of citric acid and sodium citrate, a mixture of formic acid and ammonium formate, or a combination thereof.

13. The method of claim 6, wherein the aqueous phase comprises a salt of an acid and has a pH of from 1.0 to 5.5.

14. The method of claim 13, wherein the aqueous phase has a pH of from 3.0 to 3.5.

15. The method of claim 7, wherein the buffer concentration in the aqueous phase is from 20 mM to 40 mM.

16. The method of claim 3, wherein the eluent comprises at least one organic solvent, which is acetonitrile, methanol, ethanol, isopropyl alcohol, tetrahydrofuran (THF), or a mixture thereof.

17. The method of claim 3, further comprising performing an ion exchange step.

18. The method of claim 3, wherein the cisatracurium salt is isolated from the HPLC elution liquid mixture by a Solid Phase Extraction (SPE) method, which method comprises:
contacting the HPLC elution liquid with a sorbent; and
eluting the product from the sorbent with an organic solvent.

19. The method of claim 18, wherein the SPE method further comprises:
optionally evaporating at least a portion of an organic solvent from the HPLC elution liquid;
adding an organic solvent to the HPLC elution liquid and separating the phases and optionally washing the organic layer;
optionally changing the anion using a suitable ion exchange method; and
isolating the product from the aqueous phase.

20. The method of claim 19, wherein the organic solvent added is ethyl acetate, n-propyl acetate, isopropyl acetate, n-butyl acetate, isobutyl acetate, toluene, chloroform, dichloromethane, or a mixture thereof.

21. The SPE method of claim 20, wherein the organic solvent added is dichloromethane.

22. The method of claim 3, wherein the cisatracurium salt is isolated as cisatracurium besylate having an isomeric purity of at least 98%.

23. The method of claim 22, wherein the cisatracurium salt is isolated as cisatracurium besylate having has an isomeric purity of at least 99%.

24. The method of claim 23, wherein the cisatracurium salt is isolated as cisatracurium besylate having an isomeric purity of at least 99.5%.
